(19) United States
(12) Patent Application Publication
(10) Pub. No.: US 2026/0283943 A1
(43) Pub. Date: Sep. 24, 2026

(54) STARCHLESS MOLDING PROCESS FOR ORAL PRODUCTS

(71) Applicants: Nicplas VON COSMOS, Moravian Falls, NC (US); NICOVENTURES TRADING LIMITED, London (GB)

(72) Inventors: Nicolas VON COSMOS, Moravian Falls, NC (US); Michael ZAWADZKI, Clemmons, NC (US); Matthew LAMPE, Winston-Salem, NC (US)

(21) Appl. No.: 19/469,235

(22) PCT Filed: Mar. 26, 2024
(86) PCT No.: PCT/IB2024/052900

§ 371 Date: Sep. 25, 2025

Related U.S. Application Data
(60) Provisional application No. 63/456,326, filed on Mar. 31, 2023.

(51) Int. Cl. A61K 9/00; A61K 31/00; A61K 31/465; A61K 47/26; A61K 47/36

(52) U.S. Cl. CPC A61K 9/0056; A61K 31/465; A61K 31/658; A61K 47/26; A61K 47/36

(57) ABSTRACT

Oral products in pastille form are provided herein, along with methods for the preparation thereof. The oral products include a sugar alcohol component, water, a gum binder, nicotine, and a humectant. The methods for preparing the products generally include preparing a molten sugar alcohol component, forming an aqueous mixture including water, a gum binder, nicotine, and a humectant; combining the molten sugar alcohol component with the aqueous mixture to form an oral composition; depositing the oral composition into starchless molds; and curing the oral composition in the starchless molds.

### FIELD OF THE DISCLOSURE

The present disclosure relates to flavored products intended for human use. The products are configured for oral use and deliver substances such as flavors and/or active ingredients during use. Such products may include tobacco or a product derived from tobacco, or may be tobacco-free alternatives.

### BACKGROUND

There are many categories of products intended for oral use and enjoyment. For example, oral tobacco products containing nicotine, which is known to have both stimulant and anxiolytic properties, have been available for many years. Conventional formats for so-called “smokeless” tobacco products include moist snuff, snus, and chewing tobacco, which are typically formed almost entirely of particulate, granular, or shredded tobacco, and which are either portioned by the user or presented to the user in individual portions, such as in single-use pouches or sachets. See for example, the types of smokeless tobacco formulations, ingredients, and processing methodologies set forth in U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; U.S. Pat. No. 7,810,507 to Dube et al.; U.S. Pat. No. 7,819,124 to Strickland et al.; U.S. Pat. No. 7,861,728 to Holton, Jr. et al.; U.S. Pat. No. 7,901,512 to Quinter et al.; U.S. Pat. No. 8,627,828 to Strickland et al.; U.S. Pat. No. 11,246,334 to Atchley, each of which is incorporated herein by reference.

In addition, traditional tobacco materials and non-tobacco materials have been combined with other ingredients to form product formats distinct from traditional smokeless products, with example formats including lozenges, pastilles, gels, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

There is continuing interest in the development of new types of oral products that deliver advantageous sensorial or biological activity. Such products typically contain flavorants and/or active ingredients such as nicotine, caffeine, botanicals, or cannabidiol. The format of such products can vary and include pouched products containing a powdered or granular composition, lozenges, pastilles, liquids, gels, emulsions, meltable compositions, and the like. See, for example, the types of products described in US Patent App. Pub. Nos. 2022/0160675 to Gerardi et al.; 2022/0071984 to Poole et al.; 2021/0378948 to Gerardi et al.; 2021/0330590 to Hutchens et al.; 2021/0186081 to Gerardi et al.; 2021/0177754 to Keller et al; 2021/0177043 to Gerardi et al.; 2021/0177038 to Gerardi et al.; 2021/0169867 to Holton, Jr. et al.; 2021/0169792 to Holton, Jr. et al.; 2021/0169132 to Holton, Jr. et al.; 2021/0169121 to St. Charles, and 2021/0169122 to St. Charles, each of which is incorporated herein by reference.

Oral products in the form of a pastille are conventionally made using starch molds in a process referred to as the “Mogul process.” In general, the process consists of a tray destacker, which places finished products in starch molds on a chain-driven tray conveyor. Trays are emptied, separating the molded product from starch. Empty trays are re-filled with conditioned starch, smoothed, and then stamped to produce indentations. A piston depositing system delivers molten material into the molds. The trays are stacked, and the product is allowed to dry and set. The starch is reused in the process, but first it must be cleaned, dried, and otherwise reconditioned. The starch serves several purposes in the process, including reducing the tendency of the deposited material to stick to the mold, and absorbing moisture from the deposited material, which impacts the texture of the resulting pastille. Use of starch molding has certain disadvantages, including relatively high labor and storage costs associated with its use.

### BRIEF SUMMARY

The present disclosure generally provides oral products and processes for preparing such oral products. The products are intended to impart a taste when used orally, and typically also deliver active ingredients to the consumer, such as nicotine. Such products may also impart desirable organoleptic properties when inserted into the oral cavity of a user of these products.

Accordingly, in one aspect, the disclosure provides a method of preparing an oral product in the form of a pastille, the method comprising:
preparing a molten sugar alcohol component;
forming an aqueous mixture comprising water, a gum binder, nicotine, and a humectant;
combining the molten sugar alcohol component with the aqueous mixture to form an oral composition;
depositing the oral composition into one or more starchless molds; and
curing the oral composition in the one or more starchless molds to provide the oral product.

In some embodiments, a firmness of the oral product after curing is at least 1200 grams of force when measured using a compression method on a texture analyzer under the following conditions: 3.00 mm/s Pre-Test speed, 10.00 mm/s Test Speed, 20.00 mm/s Post-Test Speed, and 100.0 g Force. In some embodiments, the firmness of the oral product after curing is in a range from about 1400 to about 2000 grams.

In some embodiments, the oral composition, prior to deposition, comprises water in an amount by weight in a range from about 8 to about 16% based on the total weight of the oral composition.

In some embodiments, the method further comprises maintaining the oral composition at a temperature in a range from about 60° C. to about 80° C. prior to the depositing. In some embodiments, curing comprises drying the oral composition for a period of time in a range from about 24 to about 72 hours, at a temperature in a range from about 20 to about 60° C., and at a relative humidity in a range from about 0 to about 45%. In some embodiments, curing comprises drying the oral composition for up to about 72 hours at a temperature above about 20° C., and at a relative humidity in a range from about 8.5 to about 35%. In some embodiments, curing comprises drying the oral composition for up to about 72 hours at a temperature in a range from about 20 to about 35° C., and at a relative humidity in a range from about 0 to about 25%. In some embodiments, curing comprises drying the oral composition for up to about 72 hours at a temperature in a range from about 30 to about 40° C., and at a relative humidity in a range from about 10 to about 35%. In some embodiments, curing comprises drying the oral composition for up to about 72 hours at a temperature in a range from about 50 to about 60° C., and at a relative humidity in a range from about 20 to about 45%.

In some embodiments, preparing the molten sugar alcohol component comprises heating a sugar alcohol component to a temperature in a range from about 160° C. to about 190° C.

In some embodiments, the sugar alcohol component comprises erythritol, arabitol, ribitol, isomalt, maltitol, dulcitol, iditol, mannitol, xylitol, lactitol, sorbitol, or a combination thereof. In some embodiments, the sugar alcohol component is a mixture of isomalt, maltitol, and erythritol. In some embodiments, the sugar alcohol component is a mixture of: isomalt in an amount by weight in a range from about 75% to about 85%, based on the total weight of the sugar alcohol mixture; maltitol in an amount by weight in a range from about 12% to about 19%, based on the total weight of the sugar alcohol mixture; and erythritol in an amount by weight in a range from about 3% to about 6%, based on the total weight of the sugar alcohol mixture.

In some embodiments, the gum binder is selected from the group consisting of gum arabic, xanthan gum, guar gum, ghatti gum, gum tragacanth, karaya gum, locust bean gum, gellan gum, and combinations thereof. In some embodiments, the gum binder is gum arabic.

In some embodiments, the humectant is added in an amount from about 2 to about 4% by weight, based on the total weight of the oral product. In some embodiments, the humectant is glycerol.

In some embodiments, the aqueous mixture further comprises an organic acid, an alkali metal salt of an organic acid, or a combination of an organic acid and an alkali metal salt of an organic acid. In some embodiments, the organic acid is an alkyl carboxylic acid, an aryl carboxylic acid, an alkyl sulfonic acid, an aryl sulfonic acid, or a combination of any thereof. In some embodiments, the organic acid has a log P value of from about 1.4 to about 4.5. In some embodiments, the organic acid has a log P value of from about 2.5 to about 3.5. In some embodiments, the organic acid has a log P value of from about 4.5 to about 8.0.

In some embodiments, the method further comprises adding a solubility enhancer to the aqueous mixture. In some embodiments, the solubility enhancer is glycerol or propylene glycol.

In some embodiments, the organic acid is octanoic acid, decanoic acid, benzoic acid, heptanesulfonic acid, or a combination thereof. In some embodiments, the organic acid comprises benzoic acid, a menthyl or tocopherol monoester of a dicarboxylic acid, or a combination thereof. In some embodiments, the dicarboxylic acid is selected from the group consisting of malonic acid, succinic acid, glutaric acid, adipic acid, fumaric acid, maleic acid, and combinations thereof. In some embodiments, the organic acid comprises tocopherol succinate, monomenthyl succinate, monomenthyl fumarate, monomenthyl glutarate, or a combination thereof.

In some embodiments, the oral composition comprises a combination of an organic acid and a sodium salt of an organic acid.

In some embodiments, a ratio of the organic acid to the sodium salt of the organic acid is from about 0.1 to about 10.

In some embodiments, the sodium salt of the organic acid is sodium benzoate.

In some embodiments, the oral composition comprises benzoic acid and sodium benzoate, octanoic acid and sodium octanoate, decanoic acid and sodium decanoate, or a combination thereof.

In some embodiments, at least a portion of the nicotine is associated with at least a portion of the organic acid or the alkali metal salt thereof, the association in the form of a nicotine-organic acid salt, an ion pair between the nicotine and a conjugate base of the organic acid, or both.

In some embodiments, the aqueous mixture further comprises at least one additive. In some embodiments, the at least one additive is selected from the group consisting of active ingredients, flavorants, sweeteners, buffering agents, colorants, salts, and mixtures thereof. In some embodiments, the active ingredient is selected from the group consisting of nutraceuticals, botanicals, stimulants, amino acids, vitamins, cannabinoids, cannabimimetics, terpenes, and combinations thereof.

In some embodiments, the nicotine is present in an amount of from about 0.001 to about 10% by weight of the oral product, calculated as the free base and based on the total weight of the oral product.

The disclosure includes, without limitations, the following embodiments.

Embodiment 1: A method of preparing an oral product in the form of a pastille, the method comprising:
preparing a molten sugar alcohol component;
forming an aqueous mixture comprising water, a gum binder, nicotine, and a humectant;
combining the molten sugar alcohol component with the aqueous mixture to form an oral composition;
depositing the oral composition into one or more starchless molds; and
curing the oral composition in the one or more starchless molds to provide the oral product.

Embodiment 2: The method of embodiment 1, wherein a firmness of the oral product after curing is at least 1200 grams of force when measured using a compression method on a texture analyzer under the following conditions: 3.00 mm/s Pre-Test speed, 10.00 mm/s Test Speed, 20.00 mm/s Post-Test Speed, and 100.0 g Force.

Embodiment 3: The method of embodiment 1 or 2, wherein a firmness of the oral product after curing is in a range from about 1400 to about 2000 grams.

Embodiment 4: The method of any one of embodiments 1-3, wherein the oral composition, prior to deposition, comprises water in an amount by weight in a range from about 8 to about 16% based on the total weight of the oral composition.

Embodiment 5: The method of any one of embodiments 1-4, further comprising maintaining the oral composition at a temperature in a range from about 60° C. to about 80° C. prior to the depositing.

Embodiment 6: The method of any one of embodiments 1-5, wherein curing comprises drying the oral composition for a period of time in a range from about 24 to about 72 hours, at a temperature in a range from about 20 to about 60° C., and at a relative humidity in a range from about 0 to about 45%.

Embodiment 7: The method of any one of embodiments 1-5, wherein curing comprises drying the oral composition for up to about 72 hours at a temperature above about 20° C., and at a relative humidity in a range from about 8.5 to about 35%.

Embodiment 8: The method of any one of embodiments 1-5, wherein curing comprises drying the oral composition for up to about 72 hours at a temperature in a range from about 20 to about 35° C., and at a relative humidity in a range from about 0 to about 25%.

Embodiment 9: The method of any one of embodiments 1-5, wherein curing comprises drying the oral composition for up to about 72 hours at a temperature in a range from about 30 to about 40° C., and at a relative humidity in a range from about 10 to about 35%.

Embodiment 10: The method of any one of embodiments 1-5, wherein curing comprises drying the oral composition for up to about 72 hours at a temperature in a range from about 50 to about 60° C., and at a relative humidity in a range from about 20 to about 45%.

Embodiment 11: The method of any one of embodiments 1-10, wherein preparing the molten sugar alcohol component comprises heating a sugar alcohol component to a temperature in a range from about 160° C. to about 190° C.

Embodiment 12: The method of any one of embodiments 1-11, wherein the sugar alcohol component comprises erythritol, arabitol, ribitol, isomalt, maltitol, dulcitol, iditol, mannitol, xylitol, lactitol, sorbitol, or a combination thereof.

Embodiment 13: The method of any one of embodiments 1-12, wherein the sugar alcohol component is a mixture of isomalt, maltitol, and erythritol.

Embodiment 14: The method of any one of embodiments 1-13, wherein the sugar alcohol component is a mixture of:
isomalt in an amount by weight in a range from about 75% to about 85%, based on the total weight of the sugar alcohol mixture;
maltitol in an amount by weight in a range from about 12% to about 19%, based on the total weight of the sugar alcohol mixture; and
erythritol in an amount by weight in a range from about 3% to about 6%, based on the total weight of the sugar alcohol mixture.

Embodiment 15: The method of any one of embodiments 1-14, wherein the gum binder is selected from the group consisting of gum arabic, xanthan gum, guar gum, ghatti gum, gum tragacanth, karaya gum, locust bean gum, gellan gum, and combinations thereof.

Embodiment 16: The method of any one of embodiments 1-15, wherein the gum binder is gum arabic.

Embodiment 17: The method of any one of embodiments 1-16, wherein the humectant is added in an amount from about 2 to about 4% by weight, based on the total weight of the oral product.

Embodiment 18: The method of any one of embodiments 1-17, wherein the humectant is glycerol.

Embodiment 19: The method of any one of embodiments 1-18, wherein the aqueous mixture further comprises an organic acid, an alkali metal salt of an organic acid, or a combination of an organic acid and an alkali metal salt of an organic acid.

Embodiment 20: The method of embodiment 19, wherein the organic acid is an alkyl carboxylic acid, an aryl carboxylic acid, an alkyl sulfonic acid, an aryl sulfonic acid, or a combination of any thereof.

Embodiment 21: The method of embodiment 19, wherein the organic acid has a log P value of from about 1.4 to about 4.5.

Embodiment 22: method of embodiment 19, wherein the organic acid has a log P value of from about 2.5 to about 3.5.

Embodiment 23: The method of embodiment 19, wherein the organic acid has a log P value of from about 4.5 to about 8.0.

Embodiment 24: The method of embodiment 23, further comprising adding a solubility enhancer to the aqueous mixture.

Embodiment 25: The method of embodiment 24, wherein the solubility enhancer is glycerol or propylene glycol.

Embodiment 26: The method of embodiment 19, wherein the organic acid is octanoic acid, decanoic acid, benzoic acid, heptanesulfonic acid, or a combination thereof.

Embodiment 27: The method of embodiment 19, wherein the organic acid comprises benzoic acid, a menthyl or tocopherol monoester of a dicarboxylic acid, or a combination thereof,

Embodiment 28: The method of embodiment 27, wherein the dicarboxylic acid is selected from the group consisting of malonic acid, succinic acid, glutaric acid, adipic acid, fumaric acid, maleic acid, and combinations thereof.

Embodiment 29: The method of embodiment 27, wherein the organic acid comprises tocopherol succinate, monomenthyl succinate, monomenthyl fumarate, monomenthyl glutarate, or a combination thereof.

Embodiment 30: The method of any one of embodiments 1-29, wherein the oral composition comprises a combination of an organic acid and a sodium salt of an organic acid.

Embodiment 31: The method of embodiment 30, wherein a ratio of the organic acid to the sodium salt of the organic acid is from about 0.1 to about 10.

Embodiment 32: The method of embodiment 19, wherein the sodium salt of the organic acid is sodium benzoate.

Embodiment 33: The method of embodiment 19, wherein the oral composition comprises benzoic acid and sodium benzoate, octanoic acid and sodium octanoate, decanoic acid and sodium decanoate, or a combination thereof.

Embodiment 34: The method of embodiment 19, wherein at least a portion of the nicotine is associated with at least a portion of the organic acid or the alkali metal salt thereof, the association in the form of a nicotine-organic acid salt, an ion pair between the nicotine and a conjugate base of the organic acid, or both.

Embodiment 35: The method of any one of embodiments 1-34, wherein the aqueous mixture further comprises at least one additive.

Embodiment 36: The method of embodiment 35, wherein the at least one additive is selected from the group consisting of active ingredients, flavorants, sweeteners, buffering agents, colorants, salts, and mixtures thereof.

Embodiment 37: The method of embodiment 36, wherein the active ingredient is selected from the group consisting of nutraceuticals, botanicals, stimulants, amino acids, vitamins, cannabinoids, cannabimimetics, terpenes, and combinations thereof.

Embodiment 38: The method of any one of embodiments 1-37, wherein the nicotine is present in an amount of from about 0.001 to about 10% by weight of the oral product, calculated as the free base and based on the total weight of the oral product.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only and should not be construed as limiting the disclosure.

FIG. 1 is a photograph illustrating starchless silicone molds placed on a plastic tray, the molds containing a curing, deposited pastille composition prepared according to an embodiment of the present disclosure.

FIG. 2 is a photograph illustrating a pastille prepared according to an embodiment of the present disclosure.

FIG. 3 is a photograph illustrating pastilles prepared according to an embodiment of the present disclosure.

FIG. 4 is a photograph illustrating a pastille prepared according to an embodiment of the present disclosure.

FIG. 5 is a photograph illustrating pastilles prepared according to an embodiment of the present disclosure.

FIG. 6A is a photograph illustrating pastilles prepared according to an embodiment of the present disclosure.

FIG. 6B is a photograph illustrating a control pastille prepared by a reference starch molding process.

### DETAILED DESCRIPTION

The present disclosure provides products configured for oral use and processes for preparing such oral products. Oral products as described herein may be provided in various forms and with various compositions (i.e., combinations of ingredients). The processes for preparing such oral products generally comprise depositing a composition into a starchless mold and curing the composition to form the oral product.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the mixture including water. Unless otherwise indicated, reference to “weight percent” of a composition reflects the total wet weight of the composition (i.e., including water).

Products of the present disclosure are configured for oral use and typically include a composition comprising a sugar alcohol component as a filler, water, a gum binder, and a humectant. In some embodiments, the products further include nicotine, at least one additive, or both nicotine and at least one additive. In some embodiments, the products further include an organic acid component. The term “configured for oral use” as used herein means that the product is provided in a form such that during use, saliva in the mouth of the user causes one or more of the components of the product (e.g., flavoring agents, nicotine, active ingredients, and the like) to pass into the mouth of the user. In one embodiment, the product is adapted to one or more deliver components to a user through mucous membranes in the user's mouth such that the one or more components can be absorbed through the mucous membranes in the mouth when the product is used. In some embodiments, the product may be adapted to deliver flavor components and/or active ingredients to a user in addition to nicotine.

Products configured for oral use as described herein may take various forms, including gels, pastilles, gums, chews, melts, tablets, lozenges, powders, and pouches. Certain products of the disclosure are in the form of solids. Certain products can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

### Pastille Products

In particular embodiments, products of the present disclosure may be provided in the form of a pastille-type product. The pastille-type products according to embodiments of the present disclosure may be configured for oral use and advantageously can provide different characteristics and properties upon insertion into the oral cavity of a user of that product. As used herein, the term “pastille” refers to a dissolvable oral product made by solidifying a liquid or gel composition, such as a composition that includes a gelling or binding agent, so that the final product is a hardened solid gel. In certain embodiments, the pastille products of the disclosure are characterized by sufficient cohesiveness to withstand light chewing action in the oral cavity without rapidly disintegrating. The pastille products of the disclosure typically do not exhibit a highly deformable chewing quality as found in conventional chewing gum. See, for example, the smokeless tobacco pastilles, tobacco-free pastille formulations, pastille configurations, pastille characteristics and techniques for formulating or manufacturing pastilles set forth in U.S. Pat. No. 9,204,667 to Cantrell et al.; U.S. Pat. No. 9,775,376 to Cantrell et al.; U.S. Pat. No. 10,357,054 to Marshall et al.; which are incorporated herein by reference. In some embodiments, the pastille-type products described herein can be chewed by a user to release different compounds (e.g., active ingredient, flavorant, etc.) into the user's mouth. Accordingly, in some embodiments, the products disclosed herein may be in the form of a dissolvable and lightly chewable pastille product for oral use. The mouthfeel of the pastille product preferably has a slightly chewable and dissolvable quality with a mild resilience or “bounce” upon chewing that gradually leads to greater malleability during use. In some embodiments, the pastille product is capable of lasting in the user's mouth for about 10-15 minutes until it completely dissolves. Preferably, the products do not, to any substantial degree, leave any residue in the mouth of the user thereof, and do not impart a slick, waxy, or slimy sensation to the mouth of the user.

Pastille products of the present disclosure typically include a mixture of ingredients in the form of a composition. For example, in some embodiments, the compositions provided herein may include components such as a sugar alcohol component, water, a gum binder, nicotine, an organic acid component, a humectant, and at least one additive. that combine to form a product configured for oral use. The relative amounts of the various components within the composition may vary, and typically are selected so as to provide the desired sensory and performance characteristics to the oral product. The example individual components of the composition are described herein below.

### Sugar Alcohol Component

Pastille products of the present disclosure typically include a sugar alcohol component. Sugar alcohols are particularly advantageous as components in the pastilles of the disclosure because such materials contribute some sweetness and do not disrupt the desired chewable characteristics of the final product. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). Isomalt is an equimolar mixture of two disaccharides, each composed of two sugars as follows: glucose and mannitol (α-D-glucopyranosido-1,6-mannitol); and glucose and sorbitol (α-D-glucopyranosido-1,6-sorbitol). In some embodiments, the sugar alcohol component comprises erythritol, arabitol, ribitol, isomalt, maltitol, dulcitol, iditol, mannitol, xylitol, lactitol, sorbitol, or combinations of any thereof. In some embodiments, the sugar alcohol component comprises isomalt, maltitol, erythritol, or a combination thereof.

The amount of the sugar alcohol component present in the composition can vary, but is typically greater than about 30%, and up to about 50% of the composition by weight, based on the total weight of the composition. A typical range of sugar alcohols within the composition can be for example, from about 35 or about 40, to about 45 or about 50%, by weight. In certain embodiments, the amount of sugar alcohol component is from about 35 to about 45% by weight, based on the total weight of the composition.

In some embodiments, combinations of sugar alcohols may be used in order to provide desired attributes to the oral product, to enhance processing ability, or both. The selection of appropriate sugar alcohol(s) and their relative amounts may be determined based on desired physical properties of the pastille product, the other components present (e.g., gum binder, humectant, the moisture content of the composition, and the like), and to address processing issues (e.g., fluidity, drying time, curing time, gelation, and the like) which may be encountered for any given composition. In some embodiments, the sugar alcohol component comprises a combination of two or even three sugar alcohols. In some embodiments, the sugar alcohol component comprises isomalt and maltitol. In some embodiments, the sugar alcohol component comprises isomalt, maltitol, and erythritol. In some embodiments, the sugar alcohol component is a combination of isomalt, maltitol, and erythritol.

When combinations of sugar alcohols are present in the sugar alcohol component, the amount of each individual sugar alcohol in the sugar alcohol component may vary. For example, in some embodiments, the sugar alcohol component is a mixture of isomalt in an amount by weight in a range from about 75% to about 85%, based on the total weight of the sugar alcohol mixture; maltitol in an amount by weight in a range from about 12% to about 19%, based on the total weight of the sugar alcohol mixture; and erythritol in an amount by weight in a range from about 3% to about 6%, based on the total weight of the sugar alcohol mixture.

### Gum Binder

Pastille products of the present disclosure typically include a gum binder. A gum binder (or combination of two or more gum binders) may be employed in amounts sufficient to provide the desired physical attributes and physical integrity to the pastille product. In some embodiments, the gum binder may function as a binder component. The amount of gum binder present and the particular gum(s) may vary based on the desired properties of the pastille product and the other components present in the composition. A representative amount of gum binder may make up at least about 25 percent of the total weight of the pastille composition. In certain embodiments, the gum binder(s) of the composition will be present in an amount of at least about 30 weight percent, at least about 35 weight percent, at least about 40 weight percent, at least about 45 weight percent, and up to about 50 weight percent, based on the total weight of the composition. In some embodiments, the gum binder in the composition may be present in an amount of about 30 weight percent to about 50 weight percent, based on the total weight of the composition.

In certain embodiments, the gum binder includes a natural gum. Particularly, natural gums (e.g., such as gum arabic) may be incorporated into the pastille products as a softener. As used herein, the term “natural gum” refers to polysaccharide materials of natural origin that are useful as softening agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. Advantageously, use of a natural gum as a softener provides the desired textural qualities necessary for forming pastille compositions, particularly those described herein. In some embodiments, increasing the amount of a natural gum (e.g., gum arabic) while subsequently decreasing the amount of sugar alcohol can advantageously increase softness in the resulting pastille product. In some embodiments, the gum binder is selected from the group consisting of gum arabic, xanthan gum, guar gum, ghatti gum, gum tragacanth, karaya gum, locust bean gum, gellan gum, and combinations thereof. In some embodiments, the gum binder is gum arabic.

### Water

The moisture content (e.g., water content) of the pastille product, prior to use by a consumer of the product, may vary according to the desired properties. For example, the water content of the pastille product may be provided within a specified range so as to dictate the final form of the product. The water content of the pastille products described herein, prior to use by a consumer of the product, may vary within such ranges according to the desired properties and characteristics, in addition to dictating the final form of the product. For example, pastille products typically possess a water content in the range of about 5 to about 20 weight percent, based on the total weight of the pastille product. In some embodiments, the moisture content of a pastille product, as present within a single unit of product prior to insertion into the mouth of the user, is within the range of about 5 to about 25 weight percent, such as about 8 to about 20 weight percent, or about 10 to about 15 weight percent, based on the total weight of the product unit. In some embodiments, the moisture content of a pastille product may be at least about 5 weight percent, at least about 10 weight percent, at least about 15 weight percent, or at least about 20 weight percent, based on the total weight of the product. The amount of water present in the final pastille product may vary from the amount of water present in the composition during preparation and processing. For example, the composition as initially prepared may be subject to evaporation or other drying of water from the composition to provide a water content in the pastille product which is lower than that in the composition used to prepare the pastille product.

### Nicotine Component

In some embodiments, pastille products of the present disclosure include a nicotine component. By “nicotine component” is meant any suitable form of nicotine (e.g., free base or salt) for providing oral absorption of at least a portion of the nicotine present. Various nicotinic compounds, and methods for their administration, are set forth in US Pat. Pub. No. 2011/0274628 to Borschke, which is incorporated herein by reference. As used herein, “nicotinic compound” or “source of nicotine” refers to naturally-occurring or synthetic nicotinic compound unbound from a plant material, meaning the compound is at least partially purified and not contained within a plant structure, such as a tobacco leaf. Most typically, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. In certain embodiments, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). The nicotine can be employed in virtually pure form or in an essentially pure form. For example, nicotine can be employed having a purity of greater than about 95 percent, or greater than about 98 percent, or greater than about 99 percent, on a weight basis.

In certain embodiments, a nicotine component may be included in the pastille product in free base form, salt form, as a complex, or as a solvate. Typically, the nicotine component is selected from the group consisting of nicotine free base, nicotine as an ion pair, a nicotine salt, and combinations thereof. In some embodiments, at least a portion of the nicotine is in its free base form. Depending on multiple variables (concentration, pH, nature of the organic acid, and the like), the nicotine present in the composition can exist in multiple forms, including ion paired, in solution (i.e., fully solvated), as the free base, as a cation, as a salt, or any combination thereof. In some embodiments, at least a portion of the nicotine is present as a nicotine salt. In some embodiments, at least a portion of the nicotine is present as an ion pair with at least a portion of the organic acid or the conjugate base thereof, as disclosed herein below.

Typically, the nicotine component is present in a concentration of at least about 0.001% by weight of the pastille product, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the pastille product. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the pastille product.

In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12:43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrylic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

### Ion Pairing

In some embodiments, at least a portion of the nicotine is present in the form of an ion pair. Accordingly, in some embodiments, the pastille product comprises nicotine or a nicotine component and an organic acid, an alkali metal salt of an organic acid, or a combination of an organic acid and an alkali metal salt of an organic acid, wherein at least a portion of the nicotine is associated with at least a portion of the organic acid or the alkali metal salt thereof in the form of a nicotine-organic acid salt, an ion pair between the nicotine and a conjugate base of the organic acid, or both.

Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (i.e., protonated nicotine, and the conjugate base of the organic acid). By “conjugate base” is meant the base resulting from deprotonation of the corresponding acid (e.g., benzoate is the conjugate base of benzoic acid). On average, a certain population of these ion pairs exists at any given time, although the formation and dissociation of ion pairs is continuous. In the oral products as disclosed herein, and/or upon oral use of said oral products (e.g., upon contact with saliva), the nicotine and the conjugate base of the organic acid exist at least partially in the form of an ion pair. Without wishing to be bound by theory, it is believed that such ion pairing may minimize chemical degradation of the basic amine and/or enhance the oral availability of the nicotine. At alkaline pH values (e.g., such as from about 7.5 to about 9), nicotine is largely present in the free base form, which has relatively low water solubility, and low stability with respect to evaporation and oxidative decomposition, but high mucosal availability. Conversely, at acidic pH values (such as from about 6.5 to about 4), nicotine is largely present in a protonated form, which has relatively high water solubility, and higher stability with respect to evaporation and oxidative decomposition, but low mucosal availability. According to the present disclosure, it has been found that the properties of stability, solubility, and availability of the nicotine in a composition configured for oral use can be mutually enhanced through ion pairing or salt formation of nicotine with appropriate organic acids and/or their conjugate bases. Specifically, nicotine-organic acid ion pairs of moderate lipophilicity result in favorable stability and absorption properties. Lipophilicity is conveniently measured in terms of log P, the partition coefficient of a molecule between a lipophilic phase and an aqueous phase, usually octanol and water, respectively. An octanol-water partitioning favoring distribution of a basic amine-organic acid ion pair into octanol is predictive of good absorption of the basic amine present in the composition through the oral mucosa.

As noted above, at alkaline pH values (e.g., such as from about 7.5 to about 9), nicotine is largely present in the free base form (and accordingly, a high partitioning into octanol), while at acidic pH values (such as from about 5.5 to about 4), nicotine is largely present in a protonated form (and accordingly, a low partitioning into octanol). According to the present disclosure, it has been found that an ion pair between certain organic acids (e.g., having a log P value of from about 1.4 to about 8.0. such as from about 1.4 to about 4.5, allows nicotine partitioning into octanol consistent with that predicted for nicotine partitioning into octanol at a pH of 8.4.

One of skill in the art will recognize that the extent of ion pairing in the disclosed composition, both before and during use by the consumer, may vary based on, for example, pH, the nature of the organic acid, the concentration of nicotine, the concentration of the organic acid or conjugate base of the organic acid present in the composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, as disclosed herein, the presence of ion pairing may be demonstrated through surrogate measures such as partitioning of the nicotine between octanol and water or membrane permeation of aqueous solutions of the nicotine plus organic acids and/or their conjugate bases.

### Organic Acid

In some embodiments, pastille products of the present disclosure include an organic acid, an alkali metal salt thereof, or a combination thereof. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient, such as a tobacco material).

Suitable organic acids will typically have a range of lipophilicity (i.e., a polarity giving an appropriate balance of water and organic solubility). Typically, lipophilicity of suitable organic acids, as indicated by log P, will vary between about 1 and about 12 (more soluble in octanol than in water). In some embodiments, the organic acid has a log P value of from about 3 to about 12, e.g., from about 3.0, about 3.5, about 4.0, about 4.5, about 5.0, about 5.5, about 6.0, about 6.5, about 7.0, about 7.5, or about 8.0, to about 8.5, about 9.0, about 9.5, about 10.0, about 10.5, about 11.0, about 11.5, or about 12.0. In certain embodiments, the lipophilicity of suitable organic acids, as indicated by log P, will vary between about 1.4 and about 4.5 (more soluble in octanol than in water). In some embodiments, the organic acid has a log P value of from about 1.5 to about 4.0, e.g., from about 1.5, about 2.0, about 2.5, or about 3.0, to about 3.5, about 4.0, about 4.5, or about 5.0. Particularly suitable organic acids have a log P value of from about 1.7 to about 4, such as from about 2.0, about 2.5, or about 3.0, to about 3.5, or about 4.0. In specific embodiments, the organic acid has a log P value of about 2.5 to about 3.5. In some embodiments, organic acids outside this range may also be utilized for various purposes and in various amounts, as described further herein below. For example, in some embodiments, the organic acid may have a log P value of greater than about 4.5, such as from about 4.5 to about 12.0. Particularly, the presence of certain solvents or solubilizing agents (e.g., inclusion in the composition of glycerin or propylene glycol) may extend the range of lipophilicity (i.e., values of log P higher than 4.5, such as from about 4.5 to about 12.0).

Without wishing to be bound by theory, it is believed that moderately lipophilic organic acids (e.g., log P of from about 1.4 to about 4.5) produce ion pairs with nicotine which are of a polarity providing good octanol-water partitioning of the ion pair, and hence partitioning of nicotine, into octanol versus water. As discussed above, such partitioning into octanol is predictive of favorable oral availability.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2-(4-isobutylphenyl) propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid.

Examples of suitable acids include, but are not limited to, the list of organic acids in Table 1.

TABLE 1
Non-limiting examples of suitable organic acids

| Acid Name | log(P) |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptanesulfonic acid | 2.0 |
| 1-octanesulfonic acid | 2.5 |
| 1-nonanesulfonic acid | 3.1 |
| monooctyl succinate | 2.8 |
| tocopherol succinate | 10.2 |
| monomenthyl succinate | 3 |
| monomenthyl glutarate | 3.4 |
| norbixin | 7.2 |
| ((2E,4E,6E,8E,10E,12E,14E,16E,18E)- | |
| 4,8,13,17-tetramethylicosa- | |
| 2,4,6,8,10,12,14,16,18-nonaenedioic | |
| acid) | |
| bixin | 7.5 |
| ((2E,4E,6E,8E,10E,12E,14E,16Z,18E)- | |
| 20-methoxy-4,8,13,17-tetramethyl-20- | |
| oxoicosa-2,4,6,8,10,12,14,16,18- | |
| nonaenoic acid) | |

In some embodiments, the organic acid is benzoic acid, a toluic acid, benzenesulfonic acid, toluenesulfonic acid, hexanoic acid, heptanoic acid, decanoic acid, or octanoic acid. In some embodiments, the organic acid is benzoic acid, octanoic acid, or decanoic acid. In some embodiments, the organic acid is octanoic acid.

In some embodiments, the organic acid is a mono ester of a di- or poly-acid, such as mono-octyl succinate, mono-octyl fumarate, or the like. For example, the organic acid is a mono ester of a dicarboxylic acid or a poly-carboxylic acid. In some embodiments, the dicarboxylic acid is malonic acid, succinic acid, glutaric acid, adipic acid, fumaric acid, maleic acid, or a combination thereof. In some embodiments, the dicarboxylic acid is succinic acid, glutaric acid, fumaric acid, maleic acid, or a combination thereof. In some embodiments, the dicarboxylic acid is succinic acid, glutaric acid, or a combination thereof.

In some embodiments, the alcohol forming the mono ester of the dicarboxylic acid is a lipophilic alcohol. Examples of suitable lipophilic alcohols include, but are not limited to, octanol, menthol, and tocopherol. In some embodiments, the organic acid is an octyl mono ester of a dicarboxylic acid, such as monooctyl succinate, monooctyl fumarate, or the like. In some embodiments, the organic acid is a monomenthyl ester of a dicarboxylic acid. Certain menthyl esters may be desirable in oral compositions as described herein by virtue of the cooling sensation they may provide upon use of the product comprising the composition. In some embodiments, the organic acid is monomenthyl succinate, monomenthyl fumarate, monomenthyl glutarate, or a combination thereof. In some embodiments, the organic acid is a monotocopheryl ester of a dicarboxylic acid. Certain tocopheryl esters may be desirable in oral compositions as described herein by virtue of the antioxidant effects they may provide. In some embodiments, the organic acid is tocopheryl succinate, tocopheryl fumarate, tocopheryl glutarate, or a combination thereof.

In some embodiments, the organic acid is a carotenoid derivative having one or more carboxylic acids. Carotenoids are tetraterpenes, meaning that they are produced from 8 isoprene molecules and contain 40 carbon atoms. Accordingly, they are usually lipophilic due to the presence of long unsaturated aliphatic chains, and are generally yellow, orange, or red in color. Certain carotenoid derivatives can be advantageous in oral compositions by virtue of providing both ion pairing and serving as a colorant in the composition. In some embodiments, the organic acid is 2E,4E,6E,8E,10E,12E,14E,16Z,18E)-20-methoxy-4,8,13,17-tetramethyl-20-oxoicosa-2,4,6,8,10,12,14,16,18-nonaenoic acid (bixin) or an isomer thereof. Bixin is an apocarotenoid found in annatto seeds from the achiote tree (Bixa orellana) and is the naturally occurring pigment providing the reddish orange color to annatto. Bixin is soluble in fats and alcohols but insoluble in water, and is chemically unstable when isolated, converting via isomerization into the double bond isomer, trans-bixin (β-bixin), having the structure:

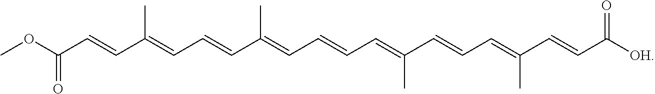 [SMILES: COC(=O)/C=C/C(C)=C/C=C/C(C)=C/C=C/C=C(C)/C=C/C=C(C)/C=C/C(=O)O]

In some embodiments, the organic acid is (2E,4E,6E,8E,10E,12E,14E,16E,18E)-4,8,13,17-tetrametbylicosa-2,4,6,8,10,12,14,16,18-nonaenedioic acid (norbixin), a water-soluble hydrolysis product of bixin having the structure:

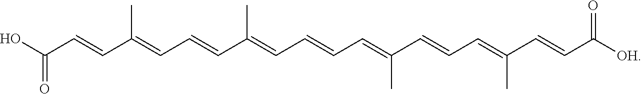 [SMILES: CC(/C=C/C=C(C)/C=C/C(=O)O)=C\C=C\C=C(C)\C=C\C=C(C)\C=C\C(=O)O]

The selection of organic acid may further depend on additional properties in addition to or without consideration to the log P value. For example, an organic acid should be one recognized as safe for human consumption, and which has acceptable flavor, odor, volatility, stability, and the like. Determination of appropriate organic acids is within the purview of one of skill in the art.

In some embodiments, more than one organic acid may be present in the pastille product. For example, the composition may comprise two, or three, or four, or more organic acids. Accordingly, reference herein to “an organic acid” contemplates mixtures of two or more organic acids. The relative amounts of the multiple organic acids may vary. For example, a pastille product may comprise equal amounts of two, or three, or more organic acids, or may comprise different relative amounts. In this manner, it is possible to include certain organic acids (e.g., citric acid or myristic acid) which have a log P value outside the desired range, when combined with other organic acids to provide the desired average log P range for the combination. In some embodiments, it may be desirable to include organic acids in the composition which have log P values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the pastille product remains below the threshold which would be found objectionable from a sensory perspective.

For example, in some embodiments, the organic acid may comprise from about 1 to about 5 or more molar equivalents of benzoic acid relative to nicotine, combined with e.g., about 0.2 molar equivalents of octanoic acid or a salt thereof, and 0.2 molar equivalents of decanoic acid or a salt thereof.

In some embodiments, the organic acid is a combination of any two organic acids selected from the group consisting of benzoic acid, a toluic acid, benzenesulfonic acid, toluenesulfonic acid, hexanoic acid, heptanoic acid, decanoic acid, and octanoic acid. In some embodiments, the organic acid is a combination of benzoic acid, octanoic acid, and decanoic acid, or benzoic and octanoic acid. In some embodiments, the pastille product comprises citric acid in addition to one or more of benzoic acid, a toluic acid, benzenesulfonic acid, toluenesulfonic acid, hexanoic acid, heptanoic acid, decanoic acid, and octanoic acid.

In some embodiments, the pastille product comprises an alkali metal salt of an organic acid. For example, at least a portion of the organic acid may be present in the pastille product in the form of an alkali metal salt. Suitable alkali metal salts include lithium, sodium, and potassium. In some embodiments, the alkali metal is sodium or potassium. In some embodiments, the alkali metal is sodium. In some embodiments, the pastille product comprises an organic acid and a sodium salt of the organic acid.

In some embodiments, the pastille product comprises benzoic acid and sodium benzoate, octanoic acid and sodium octanoate, decanoic acid and sodium decanoate, or a combination thereof.

In some embodiments, the ratio of the organic acid to the sodium salt (or other alkali metal) of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the pastille product, wherein the organic acid is added in excess of the sodium salt, in equimolar quantities with the sodium salt, or as a fraction of the sodium salt. One of skill in the art will recognize that the relative amounts will be determined by the desired pH of the pastille product, as well as the desired ionic strength. For example, the organic acid may be added in a quantity to provide a desired pH level of the pastille product, while the alkali metal (e.g., sodium) salt is added in a quantity to provide the desired extent of ion pairing. As one of skill in the art will understand, the quantity of organic acid (i.e., the protonated form) present in the pastille product, relative to the alkali metal salt or conjugate base form present in the pastille product, will vary according to the pH of the pastille product and the pKa of the organic acid, as well as according to the actual relative quantities initially added to the pastille product.

The amount of organic acid or an alkali metal salt thereof present in the pastille product, relative to nicotine, may vary. Generally, as the concentration of the organic acid (or the conjugate base thereof) increases, the percent of nicotine that is ion paired with the organic acid increases. This typically increases the partitioning of the nicotine, in the form of an ion pair, into octanol versus water as measured by the log P (the log₁₀ of the partitioning coefficient). In some embodiments, the pastille product comprises from about 0.05, about 0.1, about 1, about 1.5, about 2, or about 5, to about 10, about 15, or about 20 molar equivalents of the organic acid, the alkali metal salt thereof, or the combination thereof, relative to the nicotine component, calculated as free base nicotine.

In some embodiments, the pastille product comprises from about 2 to about 10, or from about 2 to about 5 molar equivalents of the organic acid, the alkali metal salt thereof, or the combination thereof, to nicotine, on a free-base nicotine basis. In some embodiments, the organic acid, the alkali metal salt thereof, or the combination thereof, is present in a molar ratio with the nicotine from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present.

In certain embodiments the organic acid inclusion is sufficient to provide a pastille product pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a pastille product pH of from about 4.5 to about 6.5, for example, from about 4.5, about 5.0, or about 5.5, to about 6.0, or about 6.5. In some embodiments, the organic acid is provided in a quantity sufficient to provide a pH of the pastille product of from about 5.5 to about 6.5, for example, from about 5.5, about 5.6, about 5.7, about 5.8, about 5.9, or about 6.0, to about 6.1, about 6.2, about 6.3, about 6.4, or about 6.5. In other embodiments, a mineral acid (e.g., hydrochloric acid, sulfuric acid, phosphoric acid, or the like) is added to adjust the pH of the pastille product to the desired value.

In some embodiments, the organic acid is added as the free acid, either neat (i.e., native solid or liquid form) or as a solution in, e.g., water, to the other pastille product components. In some embodiments, the alkali metal salt of the organic acid is added, either neat or as a solution in, e.g., water, to the other composition components. In some embodiments, the organic acid and the nicotine are combined to form a salt, either before addition to the composition comprising the pastille product, or the salt is formed within and is present in the pastille product as such. In other embodiments, the organic acid and nicotine are present as individual components in the pastille product and form an ion pair upon contact with moisture (e.g., saliva in the mouth of the consumer).

In some embodiments, the pastille product comprises nicotine benzoate and sodium benzoate (or other alkali metal benzoate). In other embodiments, the pastille product comprises nicotine and an organic acid, wherein the organic acid is a monoester of a dicarboxylic acid or is a carotenoid derivative having one or more carboxylic acids.

### Humectant

In some embodiments, one or more humectants may be employed in amounts sufficient to provide desired moisture attributes to the pastille product described herein. Further, in some instances, the humectant may impart desirable flow characteristics to the composition for depositing in a mold. Examples of humectants include, but are not limited to, glycerin, propylene glycol, and the like. The humectant will typically make up about 5% or less of the weight of the pastille product (e.g., from about 0.1 to about 5% by weight), for example, from about 1% to about 5% by weight, or from about 2 to about 4% by weight, based on the total weight of the pastille product.

### Additives

In some embodiments, the pastille product as disclosed herein comprises at least one additive. Suitable additives include, but are not limited to, active ingredients, flavorants, sweeteners, buffering agents, colorants, and salts. Each of these components is described further herein below.

### Active Ingredient

In some embodiments, the pastille product as disclosed herein includes one or more active ingredients. As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

In some embodiments, the active ingredient as described herein may be sensitive to degradation (e.g., oxidative, photolytic, thermal, evaporative) during processing or upon storage of the oral product. In such embodiments, the active ingredient (such as caffeine, vitamin A, and iron (Fe)) may be encapsulated, or the matrix otherwise modified with fillers, binders, and the like, to provide enhanced stability to the active ingredient. For example, binders such as functional celluloses (e.g., cellulose ethers including, but not limited to, hydroxypropyl cellulose) may be employed to enhance stability of such actives toward degradation. Additionally, encapsulated actives may need to be paired with an excipient in the composition to increase their solubility and/or bioavailability. Non-limiting examples of suitable excipients include beta-carotene, lycopene, Vitamin D, Vitamin E, Co-enzyme Q10, Vitamin K, and curcumin.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. One example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition.

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins and Minerals

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine. In some embodiments, the active ingredient comprises one or more of vitamin B6 and B12. In some embodiments, the active ingredient comprises theanine and one or more of vitamin B6 and B12.

When present, a vitamin or combination of vitamins (e.g., vitamin B6, vitamin B12, vitamin E, vitamin C, or a combination thereof) is typically at a concentration of from about 0.01% w/w to about 1% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, or about 1% by weight, based on the total weight of the composition.

In some embodiments, the active ingredient comprises vitamin A. In some embodiments, the vitamin A is encapsulated. In some embodiments, the vitamin is vitamin B6, vitamin B12, vitamin E, vitamin C, or a combination thereof.

In some embodiments, the active ingredient comprises a mineral. As used herein, the term “mineral” refers to an inorganic molecule (or related set of molecules) that is an essential micronutrient needed for the proper functioning of various systems in a mammal. Non-limiting examples of minerals include iron, zinc, copper, selenium, chromium, cobalt, manganese, calcium, phosphorus, sulfur, magnesium, and the like. In some embodiments, the active ingredient comprises iron. Suitable sources of iron include, but are not limited to, ferrous salts such as ferrous sulfate and ferrous gluconate. In some embodiments, the iron is encapsulated.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, Uva ursi, valerian, wild yam root, wintergreen, yacon root, yellow dock, Yerba mate, Yerba santa, Bacopa monniera, Withania somnifera, Lion's mane, and Silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Cannabinoids

In some embodiments, the active ingredient comprises one or more cannabinoids. As used herein, the term “cannabinoid” refers to a class of diverse natural or synthetic chemical compounds that acts on cannabinoid receptors (i.e., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD). In some embodiments, the CBD is synthetic CBD. Notably, CBD has a log P value of about 6.5, making it insoluble in an aqueous environment (e.g., saliva).

In some embodiments, the cannabinoid (e.g., CBD) is added to the oral product in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the oral product is no greater than about 1% by weight of the oral product, such as no greater than about 0.5% by weight of the oral product, such as no greater than about 0.1% by weight of the oral product, such as no greater than about 0.01% by weight of the oral product.

The choice of cannabinoid and the particular percentages thereof which may be present within the disclosed oral product will vary depending upon the desired flavor, texture, and other characteristics of the oral product.

Alternatively, or in addition to the cannabinoid, the active ingredient may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

When present, a cannabinoid (e.g., CBD) or cannabimimetic is typically in a concentration of at least about 0.1% by weight of the composition, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the composition. In some embodiments, the cannabinoid (such as CBD) is present in the oral product in a concentration of at least about 0.001% by weight of the oral product, such as in a range from about 0.001% to about 2% by weight of the oral product. In some embodiments, the cannabinoid (such as CBD) is present in the oral product in a concentration of from about 0.1% to about 1.5% by weight, based on the total weight of the oral product. In some embodiments, the cannabinoid (such as CBD) is present in a concentration from about 0.4% to about 1.5% by weight, based on the total weight of the oral product.

### Terpenes

In some embodiments, the active ingredient comprises a terpene, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl) propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Encapsulation and Stabilization of Active Ingredients

In some embodiments, the active ingredient as described herein may be sensitive to degradation (e.g., oxidative, photolytic, thermal, evaporative) during processing or upon storage of the composition. In such embodiments, the active ingredient (such as caffeine, vitamin A, and iron (Fe)) may be encapsulated, or the composition otherwise modified with suitable components (such as fillers, binders, and the like), to provide enhanced stability to the active ingredient. For example, binders such as functional celluloses (e.g., cellulose ethers including, but not limited to, hydroxypropyl cellulose) or alginate-based materials (e.g., cross linked alginate) may be employed to enhance stability of such actives toward degradation, or to provide extended and/or separate delivery of active ingredients. Additionally, encapsulated actives may need to be paired with an excipient in the composition to increase their solubility and/or bioavailability. Non-limiting examples of suitable excipients include beta-carotene, lycopene, Vitamin D, Vitamin E, Co-enzyme Q10, Vitamin K, and curcumin.

In other embodiments, in order to provide a desired concentration of the active ingredient by weight, an initial quantity of the active ingredient may be increased to compensate for a gradual degradative loss. Accordingly, larger initial amounts than those disclosed herein are contemplated by the present disclosure.

### Flavorant

In some embodiments, the pastille products comprise a flavorant. As used herein, a “flavorant” or “flavoring agent” is any flavorful or aromatic substance capable of altering the sensory characteristics associated with the pastille product of the present disclosure. Example sensory characteristics that can be modified by the flavorant include, taste, mouthfeel, moistness, coolness/heat, and/or fragrance/aroma. Non-limiting examples of flavoring agents that may be included within the present pastille product can include vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, trigeminal sensates, terpenes, and any combinations thereof. See also, Leffingwell et al., Tobacco Flavoring for Smoking Products, R. J. Reynolds Tobacco Company (1972), which is incorporated herein by reference. Flavoring agents may comprise components such as terpenes, terpenoids, aldehydes, ketones, esters, and the like. In some embodiments, the flavoring agent is a trigeminal sensate. As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form. In certain embodiments, a flavorant can be utilized in an encapsulated form such as capsules having a rupturable outer wall (e.g., a wall comprising alginate or gelatin) with an internal payload that comprises the flavorant, or flavorants encapsulated within a matrix material, such as a sugar alcohol matrix.

The flavoring agent generally comprises at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin. Flavorants are typically present in an amount of about 0.01 to about 10 weight percent, often about 0.05 to about 5 weight percent, and most often about 0.1 to about 3 weight percent, based on the total weight of the pastille product.

### Taste Modifiers

In some embodiments, in order to improve the organoleptic properties of a pastille product as disclosed herein, the pastille product may include one or more taste modifying agents (“taste modifiers”) which may serve to mask, alter, block, or improve e.g., the flavor of a pastille product as described herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the pastille product comprises an active ingredient having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., an active ingredient). Suitable taste modifiers include, but are not limited to, capsaicin, gamma-amino butyric acid (GABA), adenosine monophosphate (AMP), lactisole, or a combination thereof.

When present, a representative amount of taste modifier is about 0.01% by weight or more, about 0.1% by weight or more, or about 1.0% by weight or more, but will typically make up less than about 10% by weight of the total weight of the pastille product, (e.g., from about 0.01%, about 0.05%, about 0.1%, or about 0.5%, to about 1%, about 5%, or about 10% by weight of the total weight of the pastille product).

### Sweeteners

In some embodiments, the pastille product as disclosed herein comprises one or more sweeteners. For the avoidance of doubt, such sweeteners are in addition to the sugar alcohols present, which provide some sweetening effect to the product, but for which the primary effect is as a filler. Sweeteners can be used in natural or artificial form or as a combination of artificial and natural sweeteners. Examples of natural sweeteners include fructose, sucrose, glucose, maltose, mannose, galactose, lactose, isomaltulose, stevia, honey, and the like. Examples of artificial sweeteners include sucralose, maltodextrin, saccharin, aspartame, acesulfame K, neotame and the like. In some embodiments, the pastille product comprises an artificial sweetener. In some embodiments, the artificial sweetener is sucralose. When present, a representative amount of sweetener, whether an artificial sweetener and/or natural sugar, may make up at least about 0.01 percent or at least about 0.03 percent, of the total weight of the composition. Typically, the amount of sweetener within the pastille product will not exceed about 10 percent of the total weight of the pastille product. In some embodiments the pastille product comprises an artificial sweetener in an amount by weigh from about 0.01 to about 1 percent by weight, or from about 0.05 to about 0.1 percent by weight, based on the total weight of the pastille product.

### Salts

In some embodiments, the pastille product as disclosed herein comprises a salt. A salt (e.g., sodium chloride) may be employed in amounts sufficient to provide desired sensory attributes to the products of the present disclosure. Non-limiting examples of suitable salts include sodium chloride, potassium chloride, ammonium chloride, flour salt, and the like. When present, a representative amount of salt is at least about 0.5 weight percent or at least about 1.0 weight percent or at least about 1.5 weight percent, but will typically may make up less than about 5 percent of the total weight of the pastille product (e.g., about 0.5 to about 4, or from about 1 to about 3 weight percent).

### Colorants

In some embodiments, the pastille products described herein may include one or more colorants. A colorant may be employed in amounts sufficient to provide the desired physical attributes to the product. Examples of colorants include various dyes and pigments, such as caramel coloring and titanium dioxide. The amount of colorant utilized in the products can vary, but when present is typically up to about 3 weight percent, such as from about 0.1%, about 0.5%, or about 1%, to about 3% by weight, based on the total weight of the pastille product.

Buffering Agent and/or pH Adjuster

In some embodiments, the pastille products of the present disclosure may further comprise one or more buffering agents and/or pH adjusters (i.e., acids or bases). In such embodiments, the one or more buffering agents and/or pH adjusters are added to the composition to ensure that the final pastille product has a pH within a desirable range. It should be noted that the pH level of the oral products may be varied to alter certain characteristics of the product, for example, the release profile of the nicotine or active ingredient contained within the product. Example pH ranges for pastille products as described herein are generally from about 5 to about 7. In such embodiments, the amount of buffering agent and/or pH adjuster added to the composition is simply that amount required to bring the composition to or keep the composition at the desired pH. The amount of buffering agent and/or pH adjuster added to any given composition can be readily calculated by one skilled in the art based on the desired pH and may comprise, for example, about 0.5% to about 1% by weight of the composition. In some embodiments, the quantity of pH adjuster present may vary based on the acidity and basicity of other components which may be present in the composition (e.g., nicotine, salts, buffers, and the like). Accordingly, the buffering agent and/or pH adjuster is provided in a quantity sufficient to provide a pH of the composition of from about 5.0 to about 7.0, for example, from about 5.0, about 5.5, or about 6.0, to about 6.5, or about 7.0. In some embodiments, the buffering agent and/or pH adjuster is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5.

Additionally, various food-grade buffering agents are known and can be used to adjust the pH of the products as described herein. Suitable buffering agents may include those selected from the group consisting of acetates, glycinates, phosphates, glycerophosphates, citrates such as citrates of alkaline metals, carbonates, hydrogen carbonates, and borates, and mixtures thereof.

### Other Additives

In some embodiments, the pastille products as described herein may comprise various other additives. For example, excipients such as fillers or carriers for active ingredients (e.g., calcium polycarbophil, microcrystalline cellulose, hydroxypropylcellulose, sodium carboxymethylcellulose, cornstarch, silicon dioxide, calcium carbonate, lactose, and starches including potato starch, maize starch, etc.), thickeners, film formers and binders (e.g., hydroxypropyl cellulose, hydroxypropyl methylcellulose, acacia, sodium alginate, xanthan gum and gelatin), antiadherents (e.g., talc), glidants (e.g., colloidal silica), preservatives and antioxidants (e.g., sodium benzoate and ascorbyl palmitate), surfactants (e.g., polysorbate 80), and lubricants or processing aids (e.g., calcium stearate or magnesium stearate) may be present in the pastille product in certain embodiments. Examples of even further types of additives that may be used in the present products include thickening or gelling agents (e.g., fish gelatin), emulsifiers, oral care additives (e.g., thyme oil, eucalyptus oil, and zinc), disintegration aids, zinc or magnesium salts. Typical inclusion ranges for such additional additives can vary depending on the nature and function of the additive and the intended effect on the final product, with an example range of up to about 10% by weight, based on total weight of the pastille product (e.g., about 0.001 to about 5% by weight).

The aforementioned types of additives can be employed together (e.g., as additive formulations) or separately (e.g., individual additive components can be added at different stages involved in the preparation of the final pastille product). The relative amounts of the various components within the pastille products may vary, and typically are selected so as to provide the desired sensory and performance characteristics to the product. Furthermore, the aforementioned types of additives may be encapsulated as provided in the final product or composition. Example encapsulated additives are described, for example, in WO 2010/132444 to Atchley, which has been previously incorporated by reference herein.

### Method of Preparing Pastille Products

Provided herein are methods and processes for preparing pastille products. The manners and methods used to formulate and manufacture a pastille product as described herein above can vary. Generally, the compositions forming the pastille products are prepared such that the overall composition comprising a mixture of the various components as described herein above (e.g., water, nicotine, organic acid, sugar alcohol, gum binder, and optionally, various additives) may be used in a starchless molding process (e.g., not including a starch-based component in the molding process) for forming the pastille product. The overall composition and pastille product is generally relatively uniform in nature.

The manner by which the various components of the pastille compositions referenced above are combined may vary. The various components of the composition may be contacted, combined, or mixed together using any mixing technique or equipment known in the art. Any mixing method that brings the composition ingredients into intimate contact can be used, such as a mixing apparatus featuring an impeller or other structure capable of agitation. Examples of mixing equipment include casing drums, mixing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference.

The method comprises preparing a molten sugar alcohol component. The molten sugar alcohol component comprises at least one sugar alcohol as described herein above. In some embodiments, the at least one sugar alcohol is a mixture of isomalt with maltitol and erythritol. One or more of the sugar alcohols may be provided in the form of a sugar alcohol syrup. “Sugar alcohol syrup” as used herein is intended to refer to a viscous solution of sugar alcohol in water, e.g., having greater than about 40% solids, preferably having greater than about 50% solids, greater than about 60% solids, greater than about 70% solids, or greater than about 80% solids. Typically, the solid content of the sugar alcohol syrup primarily comprises the named sugar alcohol (e.g., maltitol syrup typically comprises greater than about 80%, greater than about 85%, or greater than about 90% by weight of maltitol on a dry weight basis). Sugar alcohol syrups are generally prepared by heating a solution of the sugar alcohol in water and cooling the mixture to give a viscous composition. The resulting syrup is typically characterized by a relatively high concentration of sugar alcohol and relatively high stability (i.e., the sugar alcohol typically does not crystallize from solution, e.g., at room temperature). Such sugar alcohol syrups can be prepared or can be obtained from commercial sources.

Preparing a molten sugar alcohol component generally comprises heating a sugar alcohol component as described herein (e.g., a mixture of sugar alcohols, such as isomalt, maltitol, and erythritol) to a temperature in a range from about 160° C. to about 190° C. for a period of time. The temperature may vary but is generally sufficient to achieve a hard crack stage. By “hard crack stage” is meant a drop of the molten sugar syrup in cold water will form hard, brittle threads that break when bent. Generally, at the hard crack stage, the majority of the water has been evaporated from the syrup (e.g., about 1% or less water remaining). In some embodiments, the sugar alcohol component may be heated to a temperature of at least about 160° C., at least about 170° C., at least about 180° C., or at least about 190° C. In some embodiments, the sugar alcohol component may be heated to a temperature of 177° C. In some instances, the heated sugar alcohol component may be allowed to cool to a temperature in the range of about 120° C. to about 160° C. prior to addition to combination with the remaining composition components. In some embodiments, for example, the heated sugar alcohol component may be cooled to a temperature of about 160° C. or less, about 150° C. or less, about 140° C. or less, or about 130° C. or less prior to combination with the remaining composition components.

The method comprises forming an aqueous mixture comprising the remaining composition components as described herein (e.g., nicotine, organic acid and/or alkali metal salt thereof, gum binder, humectant, additive) in water. In some embodiments, the gum binder (e.g., gum Arabic) is provided as a commercially solution in water. In some embodiments, the method comprises heating a gum binder, and optionally hydrating the gum binder in water. Generally, the gum binder and water may be heated to a temperature in the range of about 60° C. to about 80° C. for a period of a few seconds to a few minutes. In some embodiments, the gum binder is present in water as a solution having a gum binder percent by weight from about 40 to about 60, or about 50. In some embodiments, the gum binder solution is further diluted with additional water. The amount of water present in the gum binder solution, and the composition as a whole, may vary depending on the individual components and desired product properties. Water content of the composition and pastille product are described further herein above.

In some embodiments, the aqueous mixture is formed by mixing one or more composition components (e.g., nicotine, organic acid, salts, sweeteners, humectants, emulsifiers, flavoring agents, and others) with water to form a solution, and i) adding this solution to the gum binder solution; or ii) adding the gum binder solution to this solution of components. Alternatively, in some embodiments, the one or more components may be added, individually or in various combinations, to the gum binder solution. In particular embodiments, nicotine, an organic acid, an alkali metal salt thereof, or both an organic acid and an alkali metal salt thereof, a sweetener, a humectant, a flavoring agent, and a colorant are added to the gum binder solution to form the aqueous mixture.

The molten sugar alcohol component (optionally cooled) is then combined with the aqueous mixture to form the composition. In some embodiments, the sugar alcohol component is added to the aqueous mixture. In some embodiments, the aqueous mixture is added to the sugar alcohol component. In some embodiments, the sugar alcohol component and the aqueous mixture are combined by adding both to a separate vessel simultaneously. The combining may be performed by any suitable method of effectively mixing the components to form a homogenous composition. In some embodiments, the mixture is stirred using, for example, a high shear mixer or a bowl mixer (e.g., Hobart mixer) with a whipping attachment. In some embodiments, the composition is in the form of a solution. In some embodiments, the composition which may be in the form of a slurry. During the mixing, following the mixing, or both, the pastille composition may be heated to an elevated temperature, or an elevated temperature may be maintained for a period of time. For example, in some embodiments, the composition is heated to or maintained at a temperature in a range about 40° C. to about 80° C., such as from about 60 to about 75, or from about 70 to about 75° C., for a period of time. Such heating may be performed in order to dissolve any remaining dry ingredient within the pastille composition, to maintain a relatively fluid consistency of the composition, or both.

In some embodiments, the pastille composition, in the form of a slurry, may optionally be put through a deaerating step or process prior to being received in a mold or being subjected to other processing steps, so as to reduce or eliminate air bubbles present in the slurry mixture. Air bubbles entrapped within the slurry may affect the final weight of the pastille product, which could lead to a lack of weight uniformity between units of the final product. As such, any deaerating methods and systems may be employed for removing such air bubbles from the slurry material. For example, the slurry may be placed under reduced pressure (i.e., below atmospheric pressure) to pull the air bubbles out of the slurry mixture. In some instances, a vacuum deaerating process may be employed in which the slurry mixture is placed in a vacuum deaerator for deaerating the slurry mixture using pressure reduction. In some instances, the slurry mixture may be under vacuum for about 1 to about 10 minutes, and typically for about 3 to about 5 minutes. The deaerating step may be observed and adjusted accordingly in order to controllably remove the gaseous components from the slurry mixture.

The viscosity of the heated and deaerated slurry mixture may be measured using, for example, a Brookfield viscometer HA Series, SC4 water jacket, 27/13R sample chamber and a No. 27 spindle. The pastille composition may have a viscosity of about 5.7 Pascal-seconds (Pa·s) to about 6.2 Pa·s when heated to a temperature of about 38° C., about 4.9 Pa·s to about 5.4 Pa·s when heated to a temperature of about 43° C., and about 4.2 Pa·s to about 4.7 Pa·s when heated to a temperature of about 50° C. In some instances, extra water may be added to the pastille composition so as to provide a desired viscosity thereof.

Once the desired viscosity is achieved, the composition may then be deposited into starchless (e.g., silicone) molds. In some embodiments, the molds are held at an elevated temperature. In some embodiments, the molds are held at a temperature in a range from about 40° C. to about 80° C. (e.g., at least about 40° C. or at least about 50° C.), and typically at about 71° C.

The pastille composition is then allowed to cure and solidify into pastille form, while driving the moisture content of the pastille composition to a desired final moisture level. At this point, the pastille composition typically has a moisture content of at least about 40 percent by weight water, based on the total weight of the composition. As noted above, in some embodiments, the desired final moisture content of the pastille product may be within a range from about 5 to about 25 weight percent, or about 8 to about 20 weight percent, or about 10 to about 15 weight percent, based on the total weight of the pastille product unit. In this regard, curing generally refers to the solidification process in which moisture loss occurs, the viscosity of the composition is raised, and chemical and physical changes begin to occur (e.g., crystallization, cross-linking, gelling, film forming, etc.). The curing times and temperatures of the pastille composition may vary depending on the specific formulation of the composition, the water content, the desired product physical attributes, and the humidity to which the molds are exposed during the curing. In this regard, such variables may affect the final visual appearance of the pastille product. For example, extended curing times and/or low curing temperatures may affect the final outer configuration or contours of the pastille product. That is, the rate of drying and/or curing of the product can affect the final properties of the product. In some instances, for example, lowering the curing temperature and extending the curing time may cause the pastille product to have a relatively smooth outer surface. In contrast, curing at higher temperatures for shorter period of times can lead to a roughened or wrinkled appearance in the product.

As noted above, the temperature to which the pastille composition is exposed during drying may vary. In some embodiments, the pastille composition is dried at a temperature in a range from about 20 to about 60° C., for example, about 20, about 25, about 30, about 35, about 40, about 45, about 50, about 55, or about 60° C. In some embodiments, the pastille composition is dried at a temperature in a range from about 20 to about 30° C., such as about 25° C. In some embodiments, the pastille composition is dried at a temperature in a range from about 30 to about 60° C., such as from about 30° C. to about 40, from about 40 to about 50, or from about 50 to about 60. In some embodiments, the pastille composition is dried at a temperature of about 25° C., about 35° C., or about 60° C.

The humidity of the environment to which the pastille composition is exposed during drying may vary. In some embodiments, the pastille composition is dried at a relative humidity in a range from about 5 to about 50%, such as from about 5% to about 45%, or from about 10 to about 35%. In some embodiments, the pastille composition is dried at a relative humidity in a range from about 5 to about 10%, from about 20 to about 30%, or from about 30 to about 40%. In some embodiments, the pastille composition is dried at a relative humidity of about 8.5%. In some embodiments, the pastille composition is dried at a relative humidity of about 20 to about 25%. In some embodiments, the pastille composition is dried at a relative humidity of about 30 to about 35%.

In some embodiments, the pastille composition is dried at a temperature in a range from about 20 to about 35° C., and at a relative humidity in a range from about 0 to about 25%. In some embodiments, the pastille composition is dried at a temperature in a range from about 30 to about 40° C., and at a relative humidity in a range from about 10 to about 35%. In some embodiments, the pastille composition is dried at a temperature in a range from about 50 to about 60° C., and at a relative humidity in a range from about 20 to about 45%.

The period of time required for drying may vary, depending on the formulation, desired product moisture content, the temperature during drying, and the humidity of the drying environment. Generally, the period of time for drying is in a range from about 20 to about 72 hours, such as about 12, about 20, about 24, about 48, or about 72 hours.

In some embodiments, the drying is performed in two or more stages, using different humidity, temperature, or both in each stage. For example, the drying may be performed first under relatively high humidity and temperature conditions, followed by one or more additional stages of drying under relatively low humidity and temperature conditions. Such multi-stage drying may aid in avoiding the formation of a skin (a region of low moisture content, such as less than about 4% moisture by weight, and low diffusivity, such as less than about 10⁻¹³ m² s⁻¹) on the pastille surface. Such skin formation is generally undesirable, as it leads to uneven drying and surface defects. In some embodiments, the multi-stage drying is performed using a temperature in a range from about 50 to about 60° C., and a relative humidity in a range from about 20 to about 45%, followed by one or more of: a temperature in a range from about 30 to about 40° C., and a relative humidity in a range from about 10 to about 35%; a temperature in a range from about 20 to about 35° C., and a relative humidity in a range from about 0 to about 25%.

Following the drying, the pastille composition is then typically allowed to post-cure for a time and at a temperature suitable to allow the composition to become equilibrated to a desired moisture, shape and form. The time and temperature can vary without departing from the invention and depend in part on the desired final characteristics of the product. The pastille composition is generally allowed to cool and thereafter removed from the mold. In one embodiment, the post-cure is conducted at ambient temperature (e.g., about 20 to about 25° C.) for at least about 20 hours after being removed from the mold. In some instances, the pastille composition may be allowed to cool at refrigerated or below ambient temperatures.

Following removal from the mold, the resultant pastille product may be provided in individual pieces weighing between about 0.5 grams to about 5 grams, although aspects of the present disclosure are not limited to such weights. The pastille product can be provided in any suitable predetermined shape or form, and most preferably is provided in the form having a general shape of a pill, pellet, tablet, coin, bead, ovoid, obloid, cube, or the like.

In some embodiments, the pastille product may be coated with a coating substance after being removed from the mold and prior to or following drying. For example, a glazing or anti-sticking coating substance, such as, for example, CAPOL 410 (available from Centerchem, Inc.), may be applied to the pastille product to provide free-flowing properties. Outer coatings can also help to improve storage stability of the pastille products of the present disclosure as well as improve the packaging process by reducing friability and dusting. Devices for providing outer coating layers to the products of the present disclosure include pan coaters and spray coaters, and particularly include the coating devices available as CompuLab 24, CompuLab 36, Accela-Cota 48 and Accela-Cota 60 from Thomas Engineering.

An example outer coating comprises a film-forming polymer, such as a cellulosic polymer, an optional plasticizer, and optional flavorants, colorants, salts, sweeteners or other additives of the types set forth herein. The coating compositions are usually aqueous in nature and can be applied using any pellet or tablet coating technique known in the art, such as pan coating. Example film-forming polymers include cellulosic polymers such as methylcellulose, hydroxypropyl cellulose (HPC), hydroxypropyl methylcellulose (HPMC), hydroxyethyl cellulose, and carboxy methylcellulose. Example plasticizers include aqueous solutions or emulsions of glyceryl monostearate and triethyl citrate.

In one embodiment, the coating composition comprises up to about 75 weight percent of a film-forming polymer solution (e.g., about 40 to about 70 weight percent based on total weight of the coating formulation), up to about 5 weight percent of a plasticizer (e.g., about 0.5 to about 2 weight percent), up to about 5 weight percent of a sweetener (e.g., about 0.5 to about 2 weight percent), up to about 10 weight percent of one or more colorants (e.g., about 1 to about 5 weight percent), up to about 5 weight percent of one or more flavorants (e.g., about 0.5 to about 3 weight percent), up to about 2 weight percent of a salt such as NaCl (e.g., about 0.1 to about 1 weight percent), and the balance water. Example coating compositions and methods of application are described in, for example, U.S. Application Publication No. 2012/0055494 to Hunt et al., which is incorporated by reference herein.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

Aspects of the present invention are more fully illustrated by the following examples, which are set forth to illustrate certain aspects of the present invention and are not to be construed as limiting thereof. Unless otherwise noted, all parts and percentages are by dry weight.

### Example 1. Nicotine Pastille Using Starchless Molding Process

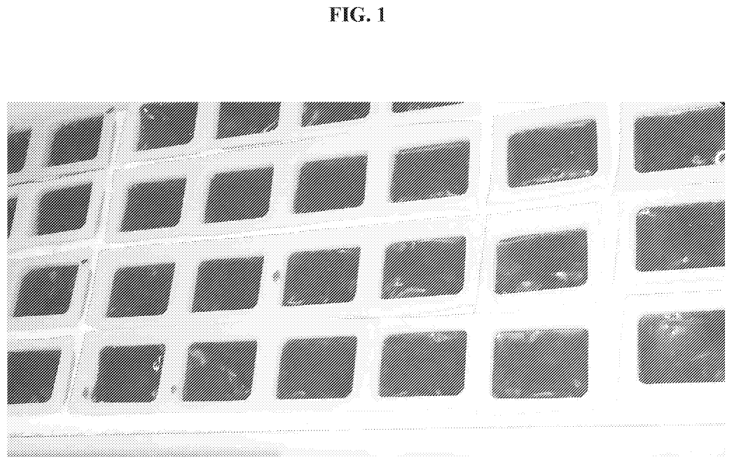

Pastilles were prepared by a starchless molding process using the formulation provided in Table 2. Isomalt, erythritol, and maltitol were mixed and heated to 177° C. (hard crack stage) to form Part A. A solution of gum Arabic (approximately 50% by weight in water) was mixed with additional water, glycerin, salt, sweetener, nicotine, sodium benzoate, color, and flavorant to form Part B. The molten Part A mixture was then mixed with (Part B) and held at 71° C. The combined mixture was deposited into silicone molds held at 71° C. (FIG. 1). The molds containing the pastille mixture were oven dried under the various conditions provided in Table 2 (drying times of 24, 48, or 72 hours, temperatures of 25, 35, or 60° C., and various relative humidity). The molds were allowed to dry under the respective condition until pastille-like products were formed (Examples 1A-1E).

TABLE 2
Starchless molded pastille composition components and quantities

| | Weight % |
|---|---|
| Part A | |
| Isomalt | 75-85 |
| Maltitol | 12-19 |
| Erythritol | 3-6 |
| Part B | |
| Gum Arabic (50% aq. solution) | 66-80 |
| Glycerin | 4-6 |
| Water | 12-15 |
| Salt | 2-4 |
| Sucralose | 0.05-0.15 |
| Nicotine | 1.5-2.5 |
| Sodium Benzoate | 0.45-0.75 |
| Color | 0.1-0.3 |
| Flavorant | 1-3 |

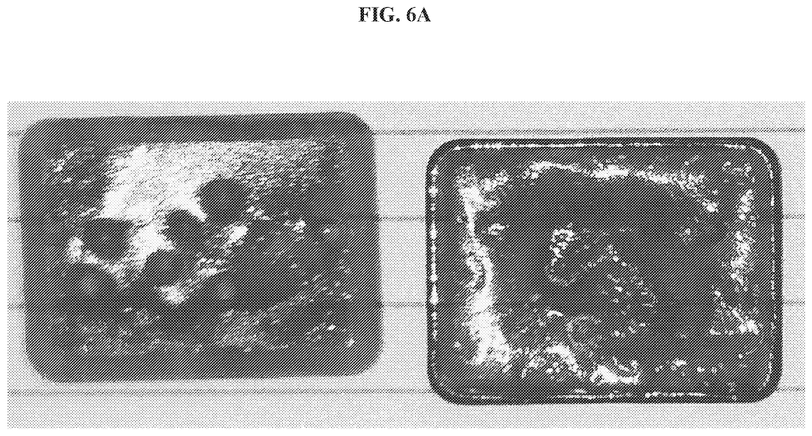

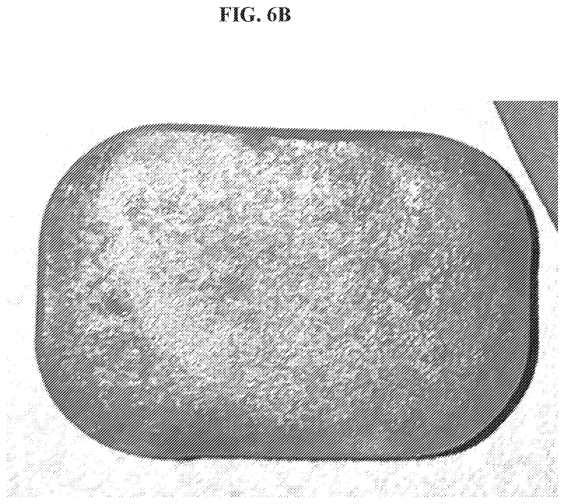

A reference pastille standard (control; FIG. 6B) was prepared similarly but using a starch molding process and the formulation of Table 3.

TABLE 3
Reference starch-molded pastille

| | Weight % |
|---|---|
| Part A | |
| Isomalt | 75-85 |
| Maltitol | 12-19 |
| Part B | |
| Gum Arabic (50% aq. solution) | 50-56 |
| Glycerin | 1.5-2.5 |
| Water | 1.2-2.1 |
| Salt | 1.4-2.6 |
| Sucralose | 0.2-0.5 |
| Nicotine | 0.4-1.2 |
| Sodium Benzoate | 0.25-0.75 |
| Color | 0.02-0.08 |
| Flavorant | 0.1-0.3 |

The firmness of the pastilles formed under each condition was evaluated and compared with using a texture analyzer (TA.XT Texture Analyzer equipped with a P/0.25S probe; Stable Micro Systems). The analyzer was programmed to analyze Force (in grams) using the Compression method (3.00 mm/s Pre-Test speed, 10.00 mm/s Test Speed, 20.00 mm/s Post-Test Speed, 100.0 g Force).

### Results

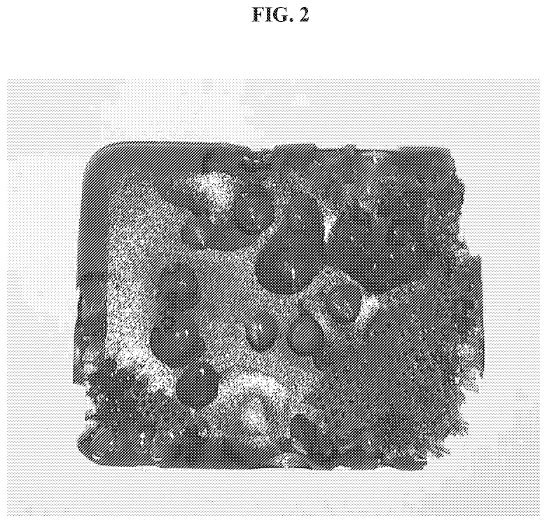

Table 4 provides the effect of temperature and humidity the time required to dry the product and in the firmness of the obtained pastille. With reference to Table 4, the different drying regimes produced pastilles with varying firmness, as well as observable differences in appearance as illustrated in FIGS. 2-6A/6B.

TABLE 4
Starchless pastille firmness, temperature, humidity and drying
time as compared to starch-molded pastille control.

| | | | Drying | Firmness | |
|---|---|---|---|---|---|
| Example | Temperature | Humidity | Time | (force, in | FIG. |
| # | (° C.) | (% rh) | (h) | grams) | # |
| 1A | 60 | 8.5 | 24 h | 106.4 | 2 |
| 1B | 35 | 22.6 | 48 h | 1956.6 | 3 |
| 1C | 25 | 47.2 | 72 h | 1500.7 | 4 |
| 1D | 25 | 35 | 72 h | 544.9 | 5 |
| 1E | 60 | 20 | 48 h | 1237.8 | 6A |
| Control | 60 | 25 | 24 h | 1808.2 | 6B |

With reference to Table 4, drying at high temperature (60° C.) with 8.5% humidity yielded pastilles with large visible bubbles and surface cracking following a 24 hour drying time (Example 1A; FIG. 2). Further, Example 1A pastilles were excessively brittle and would fall apart on touch. Without wishing to be bound by theory, it is believed that the higher temperature in combination with low humidity resulted in uneven and/or overly rapid drying, producing surface defects and poor firmness.

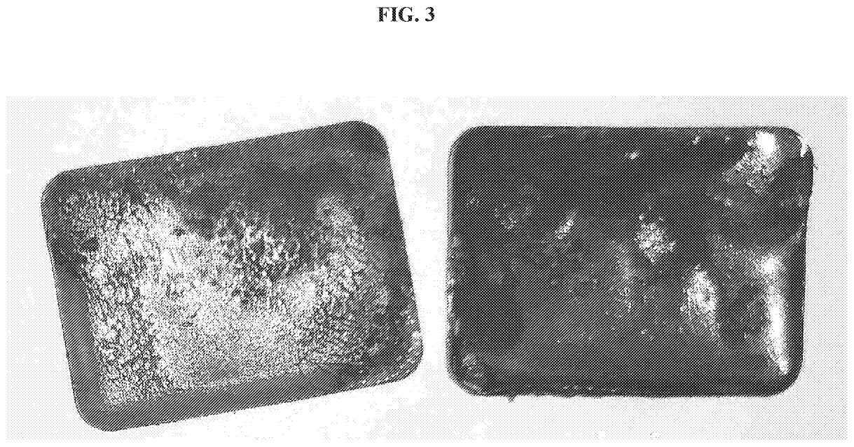

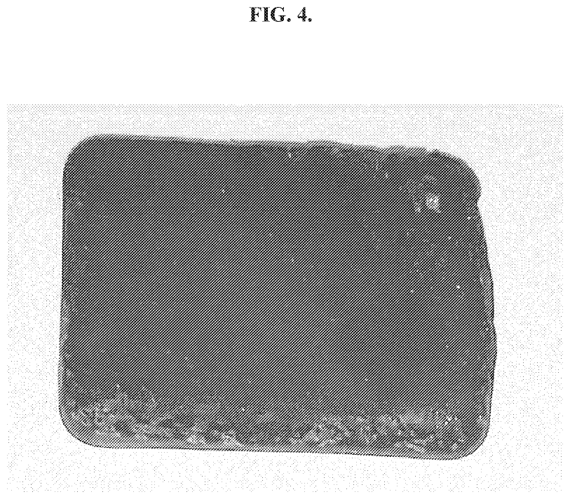

Reducing the drying temperature to 35° C. while maintaining the humidity at 22.6% (Example 1B; FIG. 3) resulted in an increase in drying time to 48 hours and produced extremely firm pastilles. Decreasing the temperature to ambient (25° C.) resulted in a further increase in drying time, but a desirable pastille firmness (Example 1C; FIG. 4).

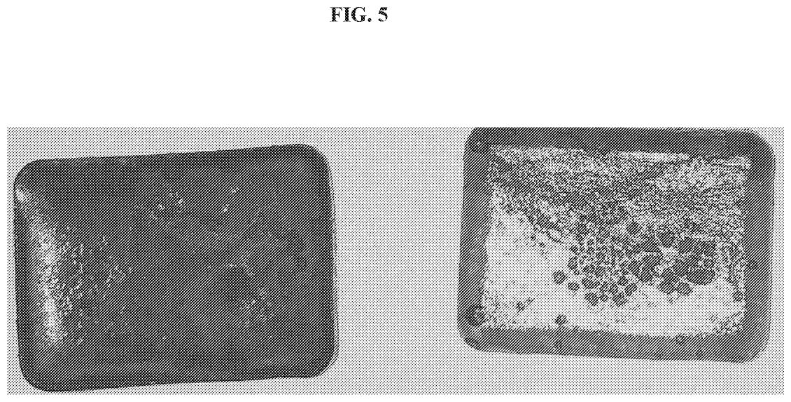

An ambient temperature of 25° C., coupled with an increase in humidity to 35% (Example 1D) also required an undesirably long (72 hour) drying time, and produced pastilles with observable defects (FIG. 5) and low firmness. Similarly, a high temperature of 60° C., coupled with a humidity of 20%, resulted in a 48-hour drying time, and produced pastilles with inadequate firmness (Example 1E) and a surface with hills and valleys (FIG. 6A).

Overall, ambient temperature (25° C.) and humidity of 35% (Example 1C) and 47% (Example 1D) resulted in smoother and flatter surfaces (FIGS. 4 and 5, respectively). With certain combinations of temperature and humidity, the pastilles were unable to achieve the desired firmness, even past the 72-hour duration of the experimental observation period (Examples 1D and 1E). Surprisingly, a small increase in temperature (to 35° C.), coupled with a reduction in humidity (22.6%), yielded a considerable gain in firmness and shortened drying time (Example 1B).

1. A method of preparing an oral product in the form of a pastille, the method comprising:
preparing a molten sugar alcohol component;
forming an aqueous mixture comprising water, a gum binder, nicotine, and a humectant;
combining the molten sugar alcohol component with the aqueous mixture to form an oral composition;
depositing the oral composition into one or more starchless molds; and
curing the oral composition in the one or more starchless molds to provide the oral product.

2. The method of claim 1, wherein a firmness of the oral product after curing is at least 1200 grams of force when measured using a compression method on a texture analyzer under the following conditions: 3.00 mm/s Pre-Test speed, 10.00 mm/s Test Speed, 20.00 mm/s Post-Test Speed, and 100.0 g Force.

3. The method of claim 1, wherein a firmness of the oral product after curing is in a range from about 1400 to about 2000 grams.

4. The method of claim 1, wherein the oral composition, prior to deposition, comprises water in an amount by weight in a range from about 8 to about 16% based on the total weight of the oral composition.

5. The method of claim 1 further comprising maintaining the oral composition at a temperature in a range from about 60° C. to about 80° C. prior to the depositing.

6. The method of claim 1, wherein curing comprises drying the oral composition for a period of time in a range from about 24 to about 72 hours, at a temperature in a range from about 20 to about 60° C., and at a relative humidity in a range from about 0 to about 45%.

7. The method claim 1, wherein curing comprises drying the oral composition for up to about 72 hours at a temperature above about 20° C., and at a relative humidity in a range from about 8.5 to about 35%.

8. The method of claim 1, wherein curing comprises drying the oral composition for up to about 72 hours at a temperature in a range from about 20 to about 35° C., and at a relative humidity in a range from about 0 to about 25%.

9. The method of claim 1, wherein curing comprises drying the oral composition for up to about 72 hours at a temperature in a range from about 30 to about 40° C., and at a relative humidity in a range from about 10 to about 35%.

10. The method of claim 1, wherein curing comprises drying the oral composition for up to about 72 hours at a temperature in a range from about 50 to about 60° C., and at a relative humidity in a range from about 20 to about 45%.

11. The method of claim 1, wherein preparing the molten sugar alcohol component comprises heating a sugar alcohol component to a temperature in a range from about 160° C. to about 190° C.

12. The method of claim 1, wherein the sugar alcohol component comprises erythritol, arabitol, ribitol, isomalt, maltitol, dulcitol, iditol, mannitol, xylitol, lactitol, sorbitol, or a combination thereof.

13. The method of claim 1, wherein the sugar alcohol component is a mixture of isomalt, maltitol, and erythritol.

14. The method of claim 1, wherein the sugar alcohol component is a mixture of:
isomalt in an amount by weight in a range from about 75% to about 85%, based on the total weight of the sugar alcohol mixture;
maltitol in an amount by weight in a range from about 12% to about 19%, based on the total weight of the sugar alcohol mixture; and
erythritol in an amount by weight in a range from about 3% to about 6%, based on the total weight of the sugar alcohol mixture.

15. The method of claim 1, wherein the gum binder is selected from the group consisting of gum arabic, xanthan gum, guar gum, ghatti gum, gum tragacanth, karaya gum, locust bean gum, gellan gum, and combinations thereof.

16. The method of claim 1, wherein the gum binder is gum arabic.

17. The method of claim 1, wherein the humectant is added in an amount from about 2 to about 4% by weight, based on the total weight of the oral product.

18. The method of claim 1, wherein the humectant is glycerol.

19. The method of claim 1, wherein the aqueous mixture further comprises an organic acid, an alkali metal salt of an organic acid, or a combination of an organic acid and an alkali metal salt of an organic acid.

20. The method of claim 19, wherein the organic acid is an alkyl carboxylic acid, an aryl carboxylic acid, an alkyl sulfonic acid, an aryl sulfonic acid, or a combination of any thereof.

21. The method of claim 19, wherein the organic acid has a log P value of from about 1.4 to about 4.5.

22. The method of claim 19, wherein the organic acid has a log P value of from about 2.5 to about 3.5.

23. The method of claim 19, wherein the organic acid has a log P value of from about 4.5 to about 8.0.

24. The method of claim 23, further comprising adding a solubility enhancer to the aqueous mixture.

25. The method of claim 24, wherein the solubility enhancer is glycerol or propylene glycol.

26. The method of claim 19, wherein the organic acid is octanoic acid, decanoic acid, benzoic acid, heptanesulfonic acid, or a combination thereof.

27. The method of claim 19, wherein the organic acid comprises benzoic acid, a menthyl or tocopherol monoester of a dicarboxylic acid, or a combination thereof.

28. The method of claim 27, wherein the dicarboxylic acid is selected from the group consisting of malonic acid, succinic acid, glutaric acid, adipic acid, fumaric acid, maleic acid, and combinations thereof.

29. The method of claim 27, wherein the organic acid comprises tocopherol succinate, monomenthyl succinate, monomenthyl fumarate, monomenthyl glutarate, or a combination thereof.

30. The method of claim 1 wherein the oral composition comprises a combination of an organic acid and a sodium salt of an organic acid.

31. The method of claim 30, wherein a ratio of the organic acid to the sodium salt of the organic acid is from about 0.1 to about 10.

32. The method of claim 19, wherein the sodium salt of the organic acid is sodium benzoate.

33. The method of claim 19, wherein the oral composition comprises benzoic acid and sodium benzoate, octanoic acid and sodium octanoate, decanoic acid and sodium decanoate, or a combination thereof.

34. The method of claim 19, wherein at least a portion of the nicotine is associated with at least a portion of the organic acid or the alkali metal salt thereof, the association in the form of a nicotine-organic acid salt, an ion pair between the nicotine and a conjugate base of the organic acid, or both.

35. The method of claim 1, wherein the aqueous mixture further comprises at least one additive.

36. The method of claim 35, wherein the at least one additive is selected from the group consisting of active ingredients, flavorants, sweeteners, buffering agents, colorants, salts, and mixtures thereof.

37. The method of claim 36, wherein the active ingredient is selected from the group consisting of nutraceuticals, botanicals, stimulants, amino acids, vitamins, cannabinoids, cannabimimetics, terpenes, and combinations thereof.

38. The method of claim 1, wherein the nicotine is present in an amount of from about 0.001 to about 10% by weight of the oral product, calculated as the free base and based on the total weight of the oral product.
